(19) United States
(12) Patent Application Publication
(10) Pub. No.: US 2002/0119139 A1
(43) Pub. Date: Aug. 29, 2002

(54) Cloning and recombinant expression of mammalian group XII secreted phospholipase A2

(76) Inventors: Michel Lazdunski, Nice (FR); Gerard Lambeau, Blausasc (FR); Emmanuel Valentin, Melun (FR)

Correspondence Address: SCHNADER HARRISON SEGAL & LEWIS, LLP, 1600 MARKET STREET, SUITE 3600, PHILADELPHIA, PA, 19103

(21) Appl. No.: 09/975,374

(22) Filed: Oct. 11, 2001

Related U.S. Application Data
(60) Provisional application No. 60/239,489, filed on Oct. 11, 2000.

(51) Int. Cl. A61K 38/46; C07H 21/04; C12N 9/16; C12P 21/02; C12N 5/06

(52) U.S. Cl. 424/94.6; 536/23.2; 435/196; 435/320.1; 435/325; 530/388.26; 435/69.1

(57) ABSTRACT

This invention concerns DNA and peptide sequences encoding a novel mammalian secreted group XII phospholipase A₂ wherein said enzyme contains a potential Ca²⁺ binding segment GCGSP and more particularly, a novel human group XII phospholipase A₂. The invention also concerns the use of this enzyme in methods for screening various chemical compounds.

### RELATED APPLICATION

This application claims the benefit of U.S. Provisional Application No. 60/239,489, filed Oct. 11, 2000. This earlier provisional application is hereby incorporated by reference.

### FIELD OF THE INVENTION

This invention relates to the cloning of a novel mammalian sPLA₂ that defines a novel group of sPLA₂s (group XII) and that is structurally distinct from the previously identified sPLA₂s. These enzymes are useful in methods for therapeutic diagnosis and screening various chemical compounds with anti-inflammatory potential or with other activities related to sPLA₂-associated functions.

### BACKGROUND

Secreted phospholipases A₂ (sPLA₂) are Ca²⁺-dependent, disulfide-rich, 14-18 kDa enzymes that catalyze the hydrolysis of phospholipids at the sn 2 position to release fatty acids and lysophospholipids (1-3). A comprehensive abbreviation system for the various sPLA₂s is used thereafter: each sPLA₂ is abbreviated with a lowercase letter indicating the sPLA₂ species (m, h, for mouse and human, respectively) followed by capital characters identifying the sPLA₂ group (GI, GII, GIII, GV, and GX) and subgroup (A, B, C, D, E, F).

In mammalian cells stimulated with proinflammatory agonists, a subset of sPLA₂s are involved in the release of arachidonic acid for eicosanoid production (4,5). The first mammalian sPLA₂ to be identified was the pancreatic sPLA₂. This sPLA₂ is found at high levels in pancreatic juice, where it has a well-known function in the digestion of dietary phospholipids (6), but also at lower levels in lung, liver, spleen, kidney, and ovary where it has been proposed to play a role in cell proliferation, acute lung injury, cell migration, and endotoxic shock (7-9). The first non-pancreatic mammalian sPLA₂ to be identified was the group IIA enzyme which is expressed at high levels during inflammation (10), and is the principal bactericidal agent against Gram-positive bacteria in human tears (11).

In addition to the above evidence, it has become clear that sPLA₂s are involved in a diverse set of physiological functions (7,12-14). In the last few years, 6 mouse and 5 human sPLA₂s structurally related to GIB and GIIA sPLA₂s (mGIIC, hGIID, mGIID, hGIIE, mGIIE, mGIIF, hGIIF, hGV, mGV, hGX, and mGX) have been identified (15-20). These group I/II/V/X sPLA₂s have similar primary structures, including identical catalytic site residues and partially overlapping sets of disulfides (21). However, they are not closely related isoforms since the level of amino acid identity is typically 20-50% among these sPLA₂s. More recently, a novel human group III sPLA₂ was identified (22), which is structurally distinct from the group I/II/V/X sPLA₂s but related to the group III sPLA₂s found in bee and lizard venoms. This novel human sPLA₂ is also disclosed in international patent application N^{o} 01/59129. This diversity of sPLA₂ structures and the fact that the tissue distribution of the different sPLA₂s are distinct argue for a diversity of physiological functions for these lipolytic enzymes.

It is also clear that mammals contain a collection of proteins that tightly bind sPLA₂s. Two types of sPLA₂ receptors (M- and N-type) and some other soluble sPLA₂ binding proteins have been identified (7,13,21,23-25) and are likely to play a role in the physiological functions of mammalian sPLA₂s and in the toxicity of a wide variety of myotoxic and neurotoxic sPLA₂s found in reptile and invertebrate venoms. Very recently, the cell surface proteoglycan glypican was also identified as a sPLA₂ binding protein able to facilitate arachidonic acid release by GIIA and GV sPLA₂s in fibroblastic cells (26).

Because of the presence of a large collection of sPLA₂s in both mammals and many reptile and invertebrate venoms, it would be highly advantageous to provide novel mammalian sPLA₂s with homology to known types of these enzymes including structurally distinct ones like the group IX sPLA₂ (Conodipine-M) from the venom of the cone snail Conus magus (27).

### SUMMARY OF THE INVENTION

This invention relates to a mammalian secreted group XII sPLA₂ containing a potential Ca²⁺ binding segment GCGSP.

### BRIEF DESCRIPTION OF THE DRAWINGS

Other advantages and characteristics of the invention will become apparent by reading the following examples concerning the cloning, genomic organization, chromosomal mapping, tissue distribution, and the enzymatic properties of the recombinant hGXII sPLA₂ in which:

FIG. 1 represents the alignment of the amino acid sequences of sPLA₂s. In panel A, the full-length sequence of hGXII is aligned with the amino acid sequences of mouse, rat, bovine and Xenopus GXII sPLA₂s (sequences were deduced from the alignment of different ESTs and from the BAC clone). For some sPLA₂s, the XX residues indicate that the sequence is partial. The Arrowhead indicates the predicted signal peptide cleavage site (32). The active site region containing catalytic site residues that are found in all sPLA₂s, and the putative Ca²⁺ binding segment GCGSP are indicated. The level of identity between the mature protein sequence of hGXII and other GXII sPLA₂s is shown. Panel B shows alignment of the Ca²⁺-binding and active site regions of hGXII with a representative member of the four other structural classes of sPLA₂s (hGIB for GI/II/V/X sPLA₂s, hGIII for GIII sPLA₂s, Conodipine-M for GIX sPLA₂, and Rice II for GXI sPLA₂s).

FIG. 2 represents a Northern blot analysis of the tissue distribution of hGXII. A commercial northern blot containing 2 μg of poly (A)⁺ RNA from different human adult tissues was hybridized at high stringency with a ³²P-labeled hGXII RNA probe as described under “Experimental Procedures.” ske. muscle, skeletal muscle; small intest., small intestine; PBL, peripheral blood leukocytes; kb, kilobase.

FIG. 3 represents the enzymatic properties of hGXII. Panel A shows initial velocity for the hydrolysis of POPC vesicles containing a small amount of 1-palmitoyl-2-[8,9-³H]palmitoyl-sn-glycero-3-phosphocholine as a function of the Ca²⁺ concentration (100,000 dpm of substrate per assay). Panel B shows initial velocity for the hydrolysis of POPG vesicles containing a small amount of 1-palmitoyl-2-[8,9-³H]palmitoyl-sn-glycero-3phosphoglycerol as a function of pH (100,000 dpm of substrate per assay). Panel C shows initial velocity for the hydrolysis of large unilamellar vesicles (0.1 μm) of the indicated phospholipid. Additional assay details have been reported elsewhere (18).

### DETAILED DESCRIPTION

This invention concerns the cloning, recombinant expression, tissue distribution, and enzymatic properties of a novel mammalian sPLA₂ and, more particularly, a novel human sPLA₂. Because this sPLA₂ clearly belongs to a new structural class, we have named it human group XII sPLA₂ (hGXII) to follow the recently identified group XI plant sPLA₂s (21,28,29).

This invention concerns the cloning, tissue distribution and recombinant expression in E. coli of a novel mammalian sPLA₂ and more particularly a novel human sPLA₂ which defines a new structural class of sPLA₂s called group XII. The human group XII (hGXII) cDNA contains a putative signal peptide of 22 residues followed by a mature protein of 167 amino acids that displays homology to known sPLA₂s only over a short stretch of amino acids in the active site region. Northern blot and RT-PCR analyses show that the tissue distribution of hGXII is distinct from the other human sPLA₂s with strong expression in heart, skeletal muscle, kidney, and pancreas and weaker expression in brain, liver, small intestine, lung, placenta, ovaries, testis, and prostate. Catalytically active hGXII was produced in E. coli and shown to be Ca²⁺-dependent despite the fact that it is predicted to have an unusual Ca²⁺ binding loop. Like for the previously characterized mouse group IIE sPLA₂s, the specific activity of hGXII is low in comparison to those of other mammalian sPLA₂, suggesting that hGXII could have novel functions that are independent of its phospholipase A₂ activity.

Thus, the invention concerns a novel mammalian secreted group XII sPLA₂ wherein said enzyme contains a potential Ca²⁺ binding segment GCGSP. The invention concerns more particularly a mammalian secreted group XII sPLA₂ comprising the sequence of amino acids under SEQ ID N^{o}2. More particularly, the mammalian secreted group XII sPLA₂ is a human secreted group XII sPLA₂.

The invention also concerns a nucleic acid molecule comprising of an encoding nucleic sequence for a mammalian secreted group XII sPLA₂ or for a fragment of a mammalian secreted group XII sPLA₂ whose amino acid sequence is SEQ ID N^{o}2. The invention relates more particularly to a nucleic acid molecule comprising the sequence under SEQ ID N^{o}1. The invention also concerns nucleotide sequences derived from the above sequence, for example, from the degeneracy of the genetic code or by the suppression or insertion of nucleotides (such as introns), and which encode for proteins presenting characteristics and properties of group XII sPLA₂.

Another aspect of the invention is polyclonal or monoclonal antibodies directed against one secreted group XII sPLA₂ of the invention, a derivative or a fragment of these. These antibodies can be prepared by the methods described in the literature. According to prior art techniques, polyclonal antibodies are formed by the injection of proteins, extracted from animal tissues or produced by genetic transformation of a host, into animals, and then recuperation of antisera and antibodies from the antiserums for example by affinity chromatography. The monoclonal antibodies can be produced by fusing myeloma cells with spleen cells from animals previously immunized using the proteins of the invention. These antibodies are useful in the search for new secreted mammalian group XII sPLA₂ or the homologues of this enzyme in other mammals or again for studying the relationship between the secreted group XII sPLA₂ of different individuals or species.

The invention also concerns a vector comprising at least one molecule of nucleic acid above, advantageously associated with adapted control sequences, together with a production or expression process in a cellular host of a mammalian group XII sPLA₂ of the invention or a fragment thereof. The preparation of these vectors as well as the production or expression in a protein host of the invention can be carried out by molecular biology and genetic engineering techniques known in the art.

An encoding nucleic acid molecule for a mammalian secreted group XII sPLA₂ or a vector according to the invention can also be used to transform animals and establish a line of transgenic animals. The vector used is chosen in function of the host into which it is to be transferred. It can be any vector such as a plasmid. Thus, the invention also relates to cellular hosts expressing mammalian secreted group XII sPLA₂ obtained in conformity with the preceding processes.

The invention also relates to nucleic and oligonucleotide probes prepared from the molecules of nucleic acid according to the invention. These probes, marked advantageously, are useful for hybridisation detection of similar group XII sPLA₂ in other individuals or species. According to prior art techniques, these probes are put into contact with a biological sample. Different hybridisation techniques can be used, such as Dot-blot hybridisation or replica hybridisation (Southern technique) or other techniques (DNA chips). Such probes constitute the tools making it possible to detect similar sequences quickly in the encoding genes for group XII sPLA₂ which allow study of the presence, origin and preservation of these proteins. The oligonucleotide probes are useful for PCR experiments, for example, to search for genes in other species or with a diagnostic aim.

The secreted phospholipases A₂ (sPLA₂) are Ca²⁺-dependent, disulfide-rich, 14-18 kDa enzymes that catalyze the hydrolysis of phospholipids at the sn-2 position to release fatty acids and lysophospholipids. sPLA₂s are also ligands that bind to a collection of soluble and membrane bound proteins which are likely to play a role in the biological functions of these enzymes. In the last few years, a number of structurally distinct mammalian sPLA₂s have been identified, and it has become clear that these sPLA₂s are expressed in a variety of tissues under both normal and pathological conditions including inflammatory diseases, cancers, cardiac and brain ischemia and the like, and are involved in a myriad of physiological and pathological roles. In mammalian cells stimulated with proinflammatory agonists, a subset of sPLA₂s play a role in the release of arachidonic acid for eicosanoid production. sPLA2s are also involved in cell proliferation, cell migration, angiogenesis, cell contraction, apoptosis, neurosecretion, blood coagulation, adipogenesis, lipid metabolism (digestion, skin lipid barrier and lung surfactant formation, lipoprotein metabolism and the like), spermatogenesis, fecondation, and embryogenesis. They also play a role in host defense and have antiviral and antibacterial properties against viruses like HIV-1 and various Gram-positive and Gram-negative bacterial strains. They also have antitumoral properties. They are also involved in various pathological conditions such as acute lung injury, acute respiratory distress syndrome, Crohn's disease, and various types of cancers where sPLA₂s can act as gene suppressors.

The invention concerns pharmaceutical compositions comprising as active agent at least an encoding nucleic acid molecule for a mammalian secreted group XII sPLA₂, or one molecule for a mammalian secreted group XII sPLA₂ or a derivative of this protein. These pharmaceutical compositions can be used to treat or prevent viral and bacterial infections. They also can be used to treat or prevent cancers.

The invention is also useful in methods for identifying biologically active compounds with anti-inflammatory properties or more generally for identifying compounds that modulate sPLA₂ biological activities as listed above.

Such biologically active compounds can be identified by determining if a selected compound is capable of inhibiting the catalytic activity of sPLA₂ in cleaving a phospholipid to release fatty acids and lysophospholipids in a mixed micelle assay, a liposome assay, a system utilizing natural membranes, or in whole cells overexpressing this enzyme. A compound capable of inhibiting sPLA₂ catalytic activity may have anti-inflammatory or may behave as an antagonist of sPLA₂ in the sPLA₂ biological activities listed above.

For example, screening of compounds for potential anti-inflammatory activity can be performed with the novel sPLA₂ enzymes of this invention, purified to homogeneity from cell sources or produced recombinantly or synthetically. A selected compound may be added to a sPLA₂ enzyme of this invention in a mixed micelle assay, a liposome assay, or an assay system utilizing natural membranes and analyzed for inhibition of sPLA₂ activity. Alternatively, a selected compound may be added to whole cells which overexpress the sPLA₂ and the cells examined for inhibition of release of fatty acids or lysophospholipids. In this case, normal cells and cells overexpressing sPLA₂ can be cultured in labeled arachidonic acid. A signal is measured between the secreted products of both the normal and overexpressing cells to provide a baseline of sPLA₂ expression. A selected compound is then added to cultures and the cultures are grown in labeled arachidonic acid. If there is a difference in the signal (e.g., the amount of arachidonic acid produced) in the cells in the presence of the compound, this compound inhibits sPLA₂ activity and may be a potential anti-inflammatory compound.

Biologically active compounds can also be identified by screening the selected compounds for their binding properties to sPLA₂ receptors that bind group XII sPLA₂s of this invention. These receptors include the family of N-type and M-type receptors which are likely to be involved in several biological activities of sPLA₂s including HIV-1 antiviral properties. For example, radioactively or fluorescently labeled sPLA₂s can be used in competition binding assays and selected compounds can be screened for inhibition of sPLA₂ binding.

Biologically active compounds can also be identified by screening the selected compounds for modulation of a sPLA₂ biological effect such as those listed above. For example, sPLA₂s of this invention may be added to cells in the presence or absence of a selected compound and cells may be assayed for cell proliferation, cell migration, cell contraction or apoptosis.

In general, another aspect of this invention is thus related to the use of a compound first identified by the methods described above. Novel pharmaceutical compositions may contain a therapeutically effective amount of a compound identified by an above method of this invention. These pharmaceutical compositions may be employed in methods for treating disease states or disorders involving group XII sPLA₂s of this invention.

I. Material and Methods.

I.1 Molecular Cloning of hGXII sPLA₂.

Searching for mammalian and venom sPLA₂ homologs in gene databases stored at the National Center for Biotechnology using the tBLASTn sequence alignment program (30) resulted in the identification of different human ESTs (Genbank BE271092, AW468813, A1189300) and a human genomic BAC clone (GenBank AC004067) that display low homology with various mammalian and venom sPLA₂s including the structurally distant conodipine-M (27). None of the ESTs were found to contain the full-length cDNA coding for the new sPLA₂ candidate, but a putative complete open reading frame could be constructed from the alignment of the different ESTs and the appropriately spliced genomic sequence. A forward primer (5′-TTT-GCG-GCC-GCA-TAT-GGA-GCT-GGC-TGC-TGC-CAA-GT; SEQ ID N^{o} 3) and a reverse primer (5′-TTT-AAG-CTT-CTA-GAA-TCT-GTC-ACT-AGC-TGT-CGG-CAT-C; SEQ ID N^{o} 4) flanking the above open reading frame and containing appropriate restriction sites were used to amplify by RT-PCR the cDNA fragment coding for hGXII sPLA₂. The expected 717 nucleotide hGXII cDNA fragment could be amplified from human fetal lung, pancreas and testis cDNAs (Clontech) using a Taq Pwo polymerase mixture (Hybaid, UK). The PCR fragments were digested with Not I and Xba I, ligated into the mammalian expression vector pRc/CMV neo (Invitrogen), and entirely sequenced. Several clones were found to be substantially identical to the consensus sequence described above.

I.2 Recombinant Expression of hGXII sPLA₂.

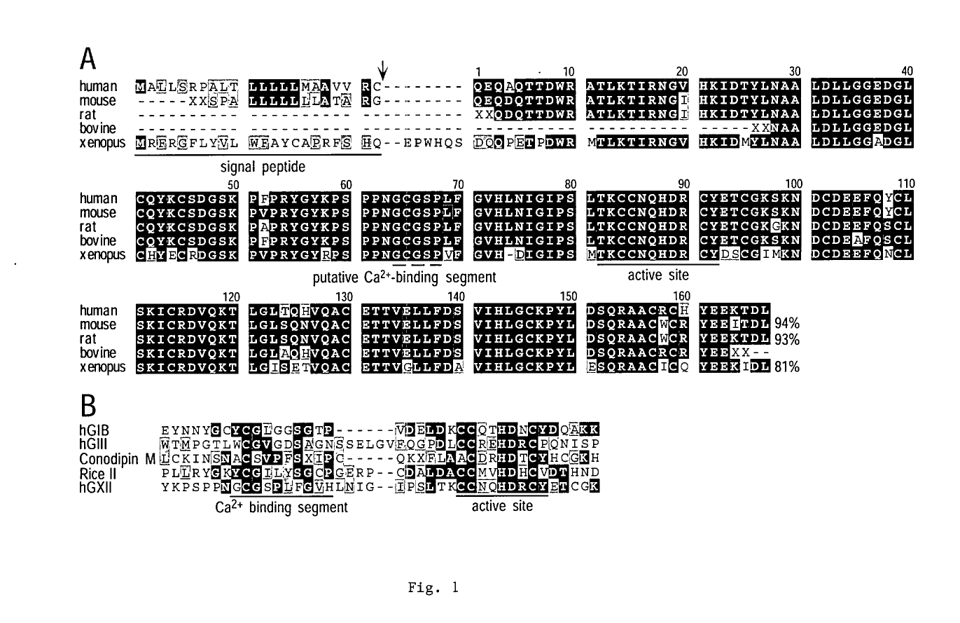

The pRc/CMVneo-hGXII construct was used as template in a PCR reaction with a forward primer (5′-TTT-GGA-TCC-ATC-GAA-GGT-CGT-CAG-GAG-CAG-GCC-CAG-ACC-GAC; SEQ ID N^{o}5), which contains a BamHI site and a factor Xa protease site (Ile-Glu-Gly-Arg) adjacent to the predicted N-terminal Gln residue of mature hGXII sPLA₂ (FIG. 1) and the reverse primer given above. The purified PCR product was digested with BamHI and HindIII and subcloned in frame with the truncated glutathione S transferase (˜10 kDa) encoded by the modified pGEX-2T vector (pAB₃), which has been previously used to express several sPLA₂s in E. coli (17). Protein production in E. coli BL21, purification of inclusion bodies, and refolding and cleavage of the fusion protein with factor Xa were carried out as described (17). Cleaved hGXII was purified by high pressure liquid chromatography on a Spherogel TSK SP-5PW column (10 μm, 0.75×7.5 cm, Altex) using a gradient of 1% acetic acid to 1 M ammonium acetate over 50 min (elution at 28 min) and was further purified on a reverse phase column (Waters RP8 Symmetry Shield, 5 μm, 100 Å, 0.46×25 cm) using a gradient of 10-60% acetonitrile in water with 0.1% trifluoroacetic acid over 50 min (elution at 36 min). The hGXII preparation appeared 100% pure when analyzed by SDS-PAGE. MALDI-TOF (Applied Biosystems DE-Pro) was carried out in the linear mode using sinapinic acid.

I.3 Analysis of the Tissue Distribution of hGXII sPLA₂.

The presence of mRNA for hGXII sPLA₂ in different human tissues was explored by northern blot and RT-PCR analyses. A human northern blot (Clontech catalog No. 7780-1) was probed as described previously (18) with a ³²P-labeled riboprobe corresponding to the hGXII coding sequence. For RT-PCR, reactions were performed with an internal forward primer (5′-GCC TTT CCC ACG TTA TGG TT; SEQ ID N^{o}6) and the reverse primer described above (200 ng each), Taq polymerase, and 1 μl of human cDNA as template (Human Multiple Tissue cDNA Panels I and II, Clontech cat. numbers K1420-1 and K1421-1). PCR was carried out at 94° C. for 2 min followed by 45 cycles of 94° C./30 sec, 60° C./30 sec, 72° C./1 min, followed by 72° C. for 5 min. PCR reactions were analyzed by Southern blotting using a [³²P]-labeled hGXII oligonucleotide probe (5′-GGA TGT GGC TCT CCA CTG TT; SEQ ID N^{o}7).

I.4 Kinetic Studies.

Large unilamellar vesicles (0.1 μm) of POPC, POPG, and POPS (31) were used to measure the initial rates of hydrolysis by hGXII in Hank's balanced salt solution with 1.2 mM CaCl₂ and 0.9 mM MgCl₂ using the fatty acid binding protein assay (17). The pH-rate profile and Ca²⁺ dependency for the action of hGXII on POPG and POPC vesicles, respectively, were obtained as described (17).

II.1 Results and Discussion.

II.1 Molecular Cloning of a Structurally Novel Human sPLA₂.

Screening of nucleic acid databases with all known types of mammalian and venom sPLA₂s (groups I, II, III, V, IX, and X) led us to identify various human ESTs and a large human BAC clone of 161,326 nucleotides coding for a putative novel sPLA₂ (hGXII) that displays homology with other sPLA₂s only in the active site region. A cDNA sequence containing a possible complete open reading frame was deduced from the alignment of the various ESTs and the genomic sequence and was then used to design primers for RT-PCR experiments with cDNA from various human tissues. The expected 717 nucleotide cDNA fragment containing an open reading frame of 567 nucleotides was amplified at a high level from human fetal lung cDNA and at lower levels from pancreas and testis cDNAs (not shown). The open reading frame was found to display some of the expected features for a sPLA₂ (FIG. 1A). The initiator methionine is followed by a 22 amino acid sequence presenting the features of a signal peptide (32) and a mature protein sequence of 167 residues. The calculated molecular mass and pI values for the mature protein are 18,702.1 Da and 6.26, respectively, and no consensus site for N-glycosylation was found. Like several other sPLA₂s, the mature hGXII sequence contains 14 cysteines and a central catalytic domain with a HD catalytic diad (FIG. 1B). Comparison of the 717 nucleotide cDNA sequence with the genomic sequence indicates that the hGXII sPLA₂ gene is composed of at least 4 exons and 3 introns spanning about 15 kilobases in length. The human BAC clone containing the hGXII gene was also found to contain different Sequence Tagged Sites positioned at the 4q25 locus, thus assigning the hGXII gene to this location on chromosome 4. Further screening of the EST databases with the hGXII cDNA sequence led to the identification of several other ESTs partially coding for mouse (GenBank AA020156 and AA204520), rat (GenBank AW918074), and bovine (GenBank AW353546) GXII sPLA₂s (FIG. 1A). A full-length amino acid sequence coding for Xenopus laevis GXII sPLA₂ was deduced from the alignment of two ESTs (GenBank AW641606 and AW639634). Interestingly, the level of identity of this novel GXII sPLA₂ among species is very high (FIG. 1A) as compared to those of other sPLA₂s (18,21).

A blastp search with the amino acid sequence of hGXII sPLA₂ against the protein databases stored at the National Center for Biotechnology reveals matches to a variety of sPLA₂s from mammals, C. elegans, plants and animal venoms, suggesting that this protein belongs to the sPLA₂ family. The homology however appears to be weak (<35% identity with blast scores lower than 35) and restricted to a short stretch of less than 60 amino acid residues containing the active site domain and the HD catalytic diad, indicating that the hGXII sPLA₂ is unique among all known sPLA₂s (FIG. 1B). The histidine of HD is thought to function as a general base to deprotonate a water molecule as it attacks the substrate ester carbonyl carbon, and the β-carboxyl group of the adjacent aspartate coordinates directly to the catalytic Ca²⁺ cofactor (6,33). Except for 3 cysteines in the active site consensus sequence CCXXHDXC which match those of other groups of sPLA₂s, the location of the other 11 cysteines residues in hGXII is distinct from that of other sPLA₂s (FIG. 1B). Since the structural arrangement of disulfides has been the main basis for designating the different sPLA₂ group numbers, the naming of the new sPLA₂ as hGXII seems appropriate.

The homology between hGXII and all known sPLA₂s is so low that it is difficult to find the Ca²⁺ binding loop, which is usually highly conserved and provides 3 of the 4 amino acid ligands for the catalytic Ca²⁺ (34). All mammalian group I, II, V, and X sPLA₂s contain 19 amino acid residues between the most N-terminal residue that serves as a ligand to the active site Ca²⁺ (i.e. His-27 of hGIIA) and the catalytic histidine (i.e. His-47 of hGIIA). In contrast, the corresponding distances for hGIII and plant GXI sPLA₂s are 25 and 23 residues, respectively. hGXII contains a potential Ca²⁺ binding segment GCGSP with 23 residues between the N-terminal glycine and the putative catalytic histidine as shown in FIG. 1. This segment is perfectly conserved among all of the GXII proteins found in gene databases. The x-ray structures of groups I, II, and III sPLA₂s reveal that the Ca²⁺ loop contains the consensus segment X₁CG₁X₂G₂. The backbone carbonyl oxygens of residues X₁, G₁, and G₂ coordinate to Ca²⁺, and the backbone NH of G₁ is proposed to donate a hydrogen bond to the carbonyl oxygen of the enzyme-susceptible substrate ester (33,35). The fact that this residue is glycine in catalytically active sPLA₂s and that mutating this residue to serine lowers catalytic activity by about 10- to 20-fold (35) argues that steric bulk is poorly tolerated at this position. The putative Ca²⁺-coordinating segment of hGXII shown in FIG. 1B fits the consensus sequence of other sPLA₂s with the exception that G₂ is a proline in hGXII. The prediction based on examination of the x-ray structures of sPLA₂s is that the hGXII Ca²⁺ binding segment should be functional. It contains GI, and the backbone carbonyl of the C-terminal proline can coordinate to Ca²⁺ since its three extra methylenes, compared to glycine, are sterically allowed because of the location of this residue on the enzyme's surface away from the substrate binding cavity. Interestingly, sPLA₂ isozymes with relatively low sPLA₂ activity from the venom of the banded krait also contain proline in place of G₂ (36).

II.2 Tissue Distribution of hGXII sPLA₂.

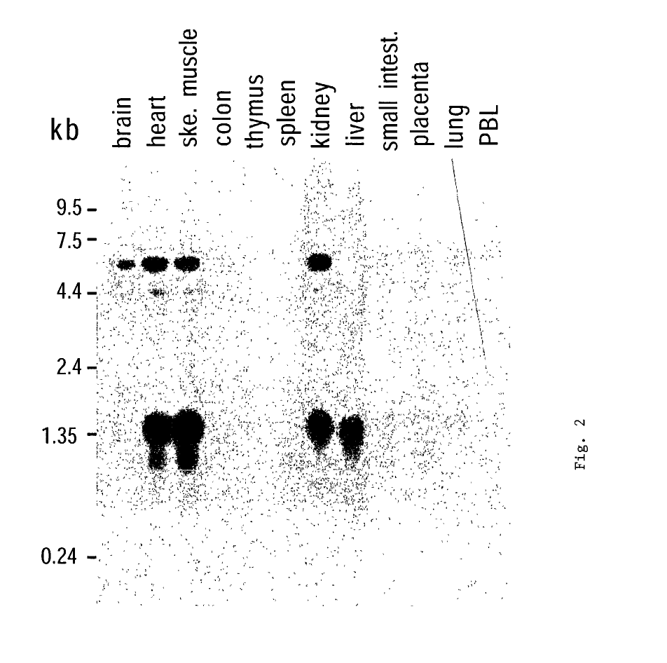

The tissue distribution of hGXII was first analyzed by hybridization at high stringency to a human northern blot (FIG. 2). hGXII is expressed as several transcripts including a major one of ˜1.4 kilobase, which is abundant in heart, skeletal muscle and kidney. hGXII transcripts are also present at lower levels in brain, liver, small intestine, lung and placenta, and expressed poorly, if at all, in colon, thymus, spleen and peripheral blood leukocytes. Furthermore, analysis by RT-PCR with commercial human tissue cDNA panels indicates a pattern of hGXII expression that is consistent with the northern blot data and additionally shows that this sPLA₂ is strongly expressed in pancreas, and weakly in ovaries, testis, and prostate (not shown). The pattern of expression of hGXII thus appears distinct from that of other known human sPLA₂s (16,19,22), suggesting specific function(s) for this novel sPLA₂.

II.3 Recombinant Expression of hGXII and Enzymatic Properties.

A mammalian expression vector containing the full-length hGXII cDNA was first used to transiently transfected HEK293 cells. The amount of sPLA₂ activity (as measured with an assay using radiolabeled E. coli membranes (16)) secreted into the culture medium 1-5 days after transfection was barely above that measured in medium from cells transfected with vector lacking the hGXII insert, suggesting that hGXII may have a low specific activity. In order to further analyze if hGXII is a catalytically active sPLA₂, we expressed hGXII as a fusion protein in E. coli, and the inclusion body fraction was submitted to a refolding strategy previously used to produce catalytically active mGIID sPLA₂ (17). After digesting the fusion protein with factor Xa protease, hGXII was purified to homogeneity by HPLC and was found to migrate as a pure protein of about 18 kDa on a Laemmli SDS gel (not shown). Mass spectrometry analysis gave an experimental mass of 18,702.6±0.5 Da, which agrees well with the mass of 18,702.1 Da calculated from the sequence of mature hGXII shown in FIG. 1A. This result indicates that all 14 cysteines are engaged in disulfide bonds, and thus it is assumed that recombinant hGXII is properly folded.

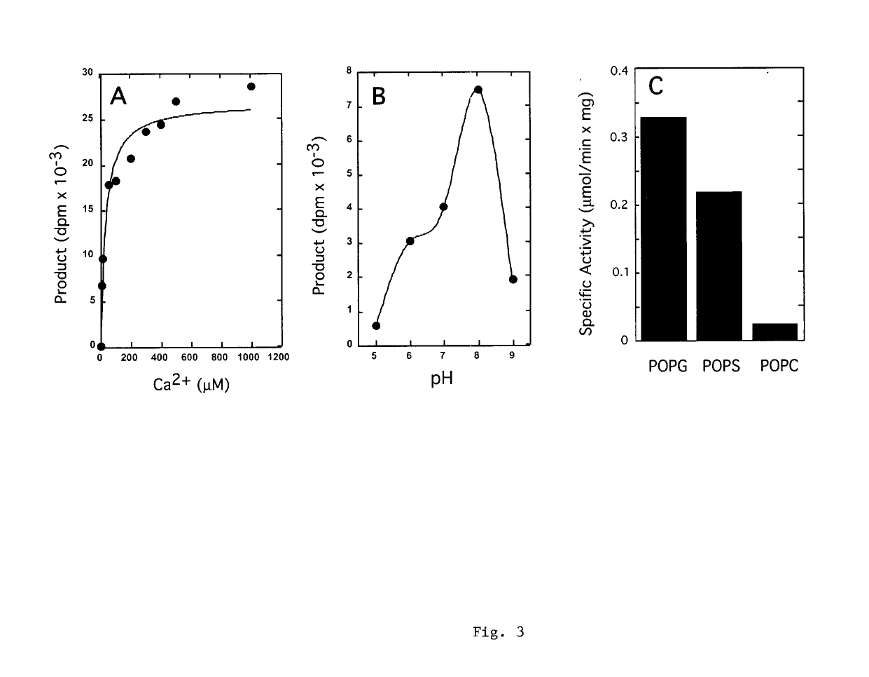

Recombinant hGXII was found to be a catalytically active sPLA₂ when assayed with the radiolabeled E. coli membrane assay (16) and with POPG, POPS, and POPC vesicles using the fatty acid binding protein assay (17). As shown in FIG. 3A, sPLA₂ activity toward POPC vesicles was strictly Ca²⁺-dependent (K_{ca}=30±10 μM). hGXII activity is maximal near pH 8.0 and decreases at higher and lower pHs (FIG. 3B). The decrease as the pH is lowered presumably reflects, in part, the protonation of the active site histidine. As for all mammalian sPLA₂s examined so far, the enzymatic activity of hGXII on phosphatidylglycerol vesicles is highest (FIG. 3C), which probably reflects the tighter binding of hGXII to anionic vesicles (37). Although hGXII hydrolyzes POPC at only ˜7% of the rate of POPG, this difference is small compared to the greater than 10⁵-fold preference of hGIIA for POPG versus POPC (18). POPS is also a good substrate for hGXII (FIG. 3C).

II.4 Concluding Remarks.

In summary, we cloned a novel catalytically active human sPLA₂, called hGXII, that belongs to a new structural class, with homologs in other mammalian species and in Xenopus laevis. Since hGXII is expressed in a limited number of human tissues and has an expression pattern distinct from those of other human sPLA₂s, it is not expected to carry out “housekeeping” functions in cells, but to have physiological function(s) distinct from those of other human sPLA₂s. A sPLA₂ gene cluster for the structurally similar hGIIA, hGIIC, hGIID, hGIIE, hGIIF, and hGV sPLA₂s is present on human chromosome 1, while structurally more distant hGIB, hGX, and hGIII sPLA₂s lie on different chromosomes (chromosomes 12, 16 and 22, respectively), as also shown in this study for hGXII sPLA₂ (chromosome 4). Recombinant expression of hGXII shows that it is a catalytically active, Ca²⁺-dependent sPLA₂. The specific enzymatic activity of hGXII appears low compared to those of other mammalian sPLA₂s (for example hGIB, hGIIA, hGV, hGX) and is comparable to the low specific activity reported for mGIIE sPLA₂ (18). This may be the reason why hGXII was not detected in earlier biochemical studies, despite the fact that this sPLA₂ is expressed in several human tissues at fairly high levels (FIG. 2). It is also interesting to note that the putative GXII sPLA₂ from zebrafish (Danio rerio) is represented in gene databases by several ESTs that all contain a leucine in place of histidine in the catalytic HD segment. This in turn suggests that either the physiological lipid substrates for these enzymes remain to be identified or that they exert their physiological functions by serving as ligands for sPLA₂ binding proteins rather than by acting as lipolytic enzymes (13).

### REFERENCES

1. Yuan, C., and Tsai, M.-D. (1999) Biochim. Biophys. Acta 1441, 215-222

2. Gelb, M. H., Jain, M. K., Hanel, A. M., and Berg, O. (1995) Ann. Rev. Biochem. 64, 653-688

3. Balsinde, J., Balboa, M. A., Insel, P. A., and Dennis, E. A. (1999) Annu. Rev. Pharmacol. Toxicol. 39, 175-189

4. Murakami, M., Shimbara, S., Kambe, T., Kuwata, H., Winstead, M. V., Tischfield, J. A., and Kudo, I. (1998) J. Biol. Chem. 273, 14411-14423

5. Balboa, M. A., Balsinde, J., Winstead, M. V., Tischfield, J. A., and Dennis, E. A. (1996) J Biol Chem 271(50), 32381-32384

6. Verheij, H. M., Slotboom, A. J., and De Haas, G. H. (1981) Rev. Physiol. Biochem. Pharmacol. 91, 91-203

7. Hanasaki, K., and Arita, H. (1999) Arch. Biochem. Biophys. 0372, 215-223

8. Rae, D., Beechey-Newman, N., Burditt, L., Sumar, N., and Hermon-Taylor, J. (1996) Scand. J. Gastroenterol. Suppl. 219, 24-27

9. Kundu, G. C., and Mukherjee, A. B. (1997) J. Biol. Chem. 272, 2346-2353

10. Pruzanski, W., Vadas, P., and Browning, J. (1993) J. Lipid Mediat. 8(3), 161-167

11. Qu, X. D., and Lehrer, R. I. (1998) Infect. Immun. 66, 2791-2797

12. Murakami, M., Nakatani, Y., Atsumi, G., Inoue, K., and Kudo, I. (1997) Crit. Rev. Immunol. 17, 225-283

13. Lambeau, G., and Lazdunski, M. (1999) Trends Pharmacol. Sci. 20, 162-170

14. Fenard, D., Lambeau, G., Valentin, E., Lefebvre, J., Lazdunski, M., and Doglio, A. (1999) J. Clin. Invest. 104, 611-618

15. Tischfield, J. A. (1997) J. Biol. Chem. 272, 17247-17250

16. Cupillard, L., Koumanov, K., Mattei, M. G., Lazdunski, M., and Lambeau, G. (1997) J. Biol. Chem. 272, 15745-15752

17. Valentin, E., Koduri, R. S., Scimeca, J.-C., Carle, G., Gelb, M. H., Lazdunski, M., and Lambeau, G. (1999) J. Biol. Chem. 274, 19152-19160

18. Valentin, E., Ghomashchi, F., Gelb, M. H., Lazdunski, M., and Lambeau, G. (1999) J. Biol. Chem. 274, 31195-31202

19. Suzuki, N., Ishizaki, J., Yokota, Y., Higashino, K., Ono, T., Ikeda, M., Fujii, N., Kawamoto, K., and Hanasaki, K. (2000) J. Biol. Chem. 275, 5785-5793

20. Ishizaki, J., Suzuki, N., Higashino, K.-C., Yokota, Y., Ono, T., Kawamoto, K., Fujii, N., Arita, H., and Hanasaki, K. (1999) J. Biol. Chem. 274, 24973-24979

21. Valentin, E., and Lambeau, G. (2000) Biochim. Biophys. Acta, 1488, 59-70

22. Valentin, E., Ghomashchi, F., Gelb, M. H., Lazdunski, M., and Lambeau, G. (2000) J. Biol. Chem. 275, 7492-7496

23. Arita, H., and Hanasaki, K. (1993) J. Lipid Mediat. 6, 217-222

24. Sartipy, P., Bondjers, G., and Hurt-Camejo, E. (1998) Arterioscler. Thromb. Vasc. Biol. 18, 1934-1941

25. Mounier, C. M., Luchetta, P., Lecut, C., Koduri, R. S., Faure, G., Lambeau, G., Valentin, E., Singer, A., Ghomashichi, F., Beguin, S., Gelb, M. H., and Bon, C. Eur. J. Biochem. 267, 4960-4969

26. Murakami, M., Kambe, T., Shimbara, S., Yamamoto, S., Kuwata, H., and Kudo, I. (1999) J. Biol. Chem. 274, 29927-29936

27. McIntosh, J. M., Ghomashchi, F., Gelb, M. H., Dooley, D. J., Stoehr, S. J., Giordani, A. B., Naisbitt, S. R., and Olivera, B. M. (1995) J. Biol. Chem. 270, 3518-3526

28. Kim, J. Y., Chung, Y. S., Ok, S. H., Lee, S. G., Chung, W. I., Kim, I. Y., and Shin, J. S. Biochim. Biophys. Acta 1489, 389-392

29. Stahl, U., Ek, B., and Stymne, S. (1998) Plant Physiol. 117, 197-205

30. Altschul, S. F., Gish, W., Miller, W., Myers, E. W., and Lipman, D. J. (1990) J.Mol. Biol. 215, 403-410

31. Bayburt, T., and Gelb, M. H. (1997) Biochemistry 36, 3216-3231

32. Nielsen, H., Engelbrecht, J., Brunak, S., and von Heijne, G. (1997) Prot. Eng. 10, 1-6

33. Scott, D. L., White, S. P., Otwinowski, Z., Yuan, W., Gelb, M. H., and Sigler, P. B. (1990) Science 250, 1541-1546

34. Scott, D. L., and Sigler, P. B. (1994) Adv. Protein. Chem. 45, 53-88

35. Bekkers, A. C., Franken, P. A., Toxopeus, E., Verheij, H. M., and de Haas, G. H. (1991) Biochim. Biophys. Acta 1076, 374-378

36. Liu, C.-S., Chen, J.-M., Chang, C.-H., Chen, S.-W., Tsai, I.-H., Lu, H.-S., and Lo, T.-B. (1990) Toxicon 28, 1457-1468

37. Jain, M. K., Ranadive, G., Yu, B.-Z., and Verheij, H. M. (1991) Biochemistry 30, 7330-7340

1) A mammalian secreted group XII sPLA₂ containing a potential Ca²⁺ binding segment GCGSP.

2) The mammalian secreted group XII sPLA₂ according to claim 1, comprising SEQ ID N^{o}2.

3) The mammalian secreted group XII sPLA₂ according to claim 1, wherein said mammalian is a human.

4) A nucleic acid molecule comprising an encoding nucleic sequence for a mammalian secreted group XII sPLA₂ or for a fragment of a mammalian secreted group XII sPLA₂ having SEQ ID N^{o}2.

5) The nucleic acid molecule according to claim 4, comprising SEQ ID N^{o}1.

6) A polyclonal or monoclonal antibody directed against a secreted group XII sPLA₂ according to claim 1, a derivative or a fragment of said antibody.

7) A vector comprising at least one molecule of nucleic acid according to claim 4, associated with adapted control sequences.

8) A cellular host transformed by one molecule of nucleic acid according to claim 4.

9) A cellular host transformed by a vector according to claim 7.

10) A nucleic and oligonucleotide probe prepared from one molecule of nucleic acid according to claim 4.

11) A pharmaceutical composition comprising as an active agent at least one nucleic acid molecule according to claim 4, or one protein according to/claim 1 or a derivative thereof.

12) A pharmaceutical composition according to claim 11 which treats and/or prevents viral and bacterial infections.

13) A pharmaceutical composition according to claim 11 which treats and/or prevents cancers.

14) A method for identifying a biologically active compound capable of inhibiting the catalytic activity of sPLA₂ according to claim 1, wherein the compound is added to a cellular host according to claim 8, and release of fatty acids and lysophospholipids is measured.

15) A method for identifying a biologically active compound for its binding properties to sPLA₂ receptors that bind group XII sPLA₂s according to claim 1 wherein a group XII sPLA₂ according to claim 1 is used in competition binding assays with said compound.

16) A method for identifying a biologically active compound modulating cell proliferation, cell migration, cell contraction or apoptosis wherein a group XII sPLA₂ according to claim 1 is added to cells in the presence or absence of said compound and cells are assayed for cell proliferation, cell migration, cell contraction or apoptosis.

17) A pharmaceutical composition containing a therapeutically effective amount of a compound identified by a method according to claim 14, for treating disease states or disorders involving group XII sPLA₂s and selected from the group consisting of inflammatory diseases, cancers, cardiac and brain ischemia, acute lung injury, acute respiratory distress syndrome and Crohn's disease.

18) A method for treating and/or preventing viral and bacterial infections in a mammal comprising administering to said mammal a therapeutically effective amount of a pharmaceutical composition according to claim 11.

19) A method for treating and/or preventing cancers in a mammal comprising administering to said mammal a therapeutically effective amount of a pharmaceutical composition according to claim 11.
